(19) United States
(12) Patent Application Publication
(10) Pub. No.: US 2008/0058831 A1
(43) Pub. Date: Mar. 6, 2008

(54) Tympanic membrane drain tube

(76) Inventor: Hisao Fujiwara, Nagasaki-shi (JP)

Correspondence Address: HARNESS, DICKEY & PIERCE, P.L.C., P.O. BOX 8910, RESTON, VA, 20195, US

(21) Appl. No.: 11/700,017

(22) Filed: Jan. 31, 2007

(30) Foreign Application Priority Data
Sep. 5, 2006 (JP) P2006-273467

(51) Int. Cl. A61F 11/00

(52) U.S. Cl. 606/109

(57) ABSTRACT

A tympanic membrane drain tube is formed so that it is possible to freely trim a tip end of a hollow tube which has a large diameter at a rear end on the external ear side and tapers toward a front end, whereby the drain tube can be inserted into a stoma formed in the tympanic membrane under clear vision, and can naturally shed.

### BACKGROUND OF THE INVENTION

1. Field of the Invention

The present invention relates to a drain tube for use in otologic surgical treatment, of which drain tube ventilation effect causes an improvement in hypoxic condition attributable to exudative otitis media, chronic ottitis media, and the like disease so that middle ear mucosa is normalized to thereby drain an accumulated middle ear exudate to outside of a middle ear cavity.

2. Description of the Related Art

When a person contracts exudative otitis media or the like disease, the person has the middle ear falling into a hypoxic condition, with the result that an exudate accumulates in the middle ear cavity, movements of the tympanic membrane and auditory ossicle become worse, and sound entering through the external ear canal becomes hard to transmit into the internal ear.

Therefore, when an exudate has accumulated in the middle ear cavity, it is necessary to incise the tympanic membrane and forcedly aspirate the exudate, thereby eliminating the exudate from the middle ear cavity and promoting ventilation of the middle ear.

After the exudate accumulated in the middle ear cavity is eliminated by incising the tympanic membrane and aspirating the exudate, a hearing level is usually recovered. However, an opening formed in the tympanic membrane spontaneously closes in a few days and therefore, in a case where the patient has not recovered completely, it often happens that an exudate accumulates again in the middle ear cavity which has been spontaneously closed, resulting in return to the former symptom.

Accordingly, a ventilation treatment for normalization of the middle ear mucosa by means of blowing air into the middle ear cavity after incision of the tympanic membrane is an important factor of a remedy for exudative otitis media and the like disease.

At present, in the ventilation treatment, an instrument having an opening for ventilation, which is called a tympanic membrane drain tube, is inserted and fixed into the tympanic membrane, whereby the middle ear cavity is allowed to be ventilated.

However, the tympanic membrane drain tube used at present, represented by a drain tube produced by Koken Co., Ltd. which is most commonly used in an operation, has flange-like portions not only on the external ear side but also on the middle ear side so that the drain tube does not come off easily during the treatment, as drain tubes 20A, 20B, 20C, and 20D illustrated in FIGS. 9 to 12.

It is true that the flange-like portions are effective for preventing the drain tube from coming off, but various problems arise in inserting the drain tube.

To be specific, since the flange-like portions of the conventional drain tube are large, it is necessary to largely incise the tympanic membrane more than necessary in order to insert the drain tube into the tympanic membrane, and a doctor needs considerable experience and skill for insertion and attachment of the drain tube to the narrow external ear canal.

Further, since the conventional drain tube is usually made of a hard material, it is difficult to process the drain tube in accordance with the symptom of the patient.

Accordingly, it takes a lot of trouble to perform the operation, and the operation inevitably takes a long time, with the result that the operation requires considerable experience and skill therefore. Furthermore, the patient is forced to bear a considerably level of invasion that involves a pain.

In view of the heavy burden on the patient and in the case where the patient is an infant who is hard to undergo an operation or in the case of presence of adhesion, not local anesthesia but general anesthesia may be employed. As a result, the patient needs to stay in hospital, and thus in many cases, the operation cannot be performed in the doctor's office.

Furthermore, a mucus plug is liable to be formed in the tube immediately after the operation, which may cause a loss of the ventilation effect. The patient therefore needs to be treated with a postoperative care.

Besides, a foreign-body reaction arising between the tube and the tympanic membrane causes inflammation which results in granulation and crust formation.

On the other hand, when the symptom subsides and the drain tube thus becomes unnecessary, the removal of the drain tube imposes a heavy burden including pain on the patient.

Therefore, in many cases, the patient has to wait for the drain tube to naturally shed for a long period of time, for example, more than 60 days even after the symptom greatly subsides or is completely cured.

### SUMMARY OF THE INVENTION

Hence, an object of the invention is to the invention provide a tympanic membrane drain tube in which the aforementioned problems to be solved of the conventional type of tympanic membrane drain tube are solved.

To be specific, the tympanic membrane drain tube according to the invention does not have a flange-like portion on the middle ear side that the conventional drain tube has, and is formed into a tapered shape as a whole.

The tympanic membrane drain tube according to the invention is formed into a shape in which the tympanic membrane drain tube can be easily pinched with a dressing forceps or the like. That is to say, the tympanic membrane drain tube has a smaller flange-like portion which is, on the external ear side, formed into an annular shape that less irritates the tympanic membrane and has a smaller contact area therewith. The tympanic membrane drain tube is formed so that a tip end thereof turns slightly upward to allow visualization of the tip and an incised portion of the tympanic membrane when the drain tube is pinched with a dressing forceps or the like.

A main body is made of a plastic material or the like, in consideration of easy and safe insertion, so as to be trimmed easily with scissors or the like in the case where the operation is hard to be performed.

The tympanic membrane drain tube according to the invention achieves effects as described below when compared with the drain tube which has been used conventionally.

First, since the tympanic membrane drain tube according to the invention does not have a flange-like portion on the middle ear side, it is possible to make an incised portion of the tympanic membrane smaller, causing no significant resistance. As a result, it is possible to shorten a length of operation time, thus leading enhancement in safety thereof.

When the drain tube according to the invention is pinched with an instrument such as a dressing forceps, the tip end of the drain tube turns slightly upward, in a consequence whereof operator's eyes, the flange-like portion of the tube, and a stoma formed in incising the tympanic membrane are not arranged on the same visual axis. The end of the tube is thus not brought under the forceps and therefore not out of sight. Accordingly, the operator can visually check the position of the tip end of the tube so that the tube can be inserted under clear vision. This can contribute to significant enhancement in safety of operation.

Moreover, the patient is not forced to bear a heavy burden during the operation, thus requiring only the minimum necessary amounts of an anesthetic and so on. Therefore, the patient can undergo the operation in the doctor's office.

Since a hollow tube is formed into a tapered shape, the drain tube naturally sheds within three weeks after the operation. In a case where a trouble occurs before the drain tube naturally sheds, the drain tube can be easily removed andn reinserted. Accordingly, it is also possible to perpetually keep the drain tube in the tympanic membrane.

Thus, a period of intubation is short. This contributes to less troubles arising in a living body, and if a trouble arises, it is easy to deal with.

Since the drain tube can be trimmed to adjust its length and so on by use of scissors or the like, it is possible to process the drain tube on the spot in accordance with the symptom of the patient, and it is also possible to cut the drain tube so as to facilitate intubation.

In the case of using a porous hollow tube, it is possible to make a larger amount of air circulate in the middle ear.

Since the drain tube according to the invention has a simple structure, it is possible to make an inner diameter of a vent hole larger than that of the conventional drain tube, and it is possible to achieve a higher ventilation effect than in the drain tube which has been used conventionally.

Even if any trouble occurs after the operation, to the drain tube can be immediately removed to eliminate the trouble. Therefore, it is possible to speedily deal with troubles. For example, even if the accumulated exudate flows into the drain tube and is then dried to form a plug, the drain tube can be removed without imposing a burden on the patient in order to clean the drain tube, and it is also easy to reinsert the drain tube.

In more detail, the invention provides a tympanic membrane drain tube comprising a hollow tube,

wherein the hollow tube is formed so as to have a large diameter at a rear end and taper toward a front end, and

wherein the hollow tube can be safely inserted into a stoma formed in a tympanic membrane under clear vision.

In the invention, it is preferable that the hollow tube is made of a plastic material and can be trimmed.

In the invention, it is preferable that a flange portion is provided at the rear end of the hollow tube.

In the invention, it is preferable that the hollow tube has a fine perforation.

In the invention, it is preferable that a tongue-like assist tool is provided at the rear end of the hollow tube.

In the invention, it is preferable that the hollow tube has a section taken along its axial line which has one long side perpendicular to its short sides and another long side inclined toward the one long side and the another long side is slanted toward a tip end so as to form an inequable tapered shape.

### BRIEF DESCRIPTION OF THE DRAWINGS

Other and further objects, features, and advantages of the invention will be more explicit from the following detailed description taken with reference to the drawings wherein:

FIG. 1 is a perspective view illustrating a tympanic membrane drain tube according to a first embodiment of the invention;

FIG. 2 is a cross sectional view illustrating the tympanic membrane drain tube according to the first embodiment of the invention;

FIG. 3 is a view for explaining an inserted state of the tympanic membrane drain tube according to the first embodiment of the invention;

FIG. 4 is a view for explaining a pinched state of the tympanic membrane drain tube according to the first embodiment of the invention;

FIG. 5 is a front view illustrating a tympanic membrane drain tube according to a second embodiment of the invention;

FIG. 6 is a front view illustrating a tympanic membrane drain tube according to a third embodiment of the invention;

FIG. 7 is a front view illustrating a tympanic membrane drain tube according to a fourth embodiment of the invention;

FIG. 8 is a front view illustrating a tympanic membrane drain tube according to a fifth embodiment of the invention;

FIG. 9 is a front view illustrating a tympanic membrane drain tube of a first conventional design;

FIG. 10 is a front view illustrating a tympanic membrane drain tube of a second conventional design;

FIG. 11 is a front view illustrating a tympanic membrane drain tube of a third conventional design; and

FIG. 12 is a front view illustrating a tympanic membrane drain tube of a fourth conventional design.

### DETAILED DESCRIPTION

Now referring to the drawings, preferred embodiments of the invention are described below.

A tympanic membrane drain tube according to the invention is formed so as to be capable of being inserted easily into a stoma formed in a tympanic membrane under clear vision by freely cutting in accordance with the symptom of the patient a tip end of a hollow tube which has a large diameter at a rear end and tapers toward a front end.

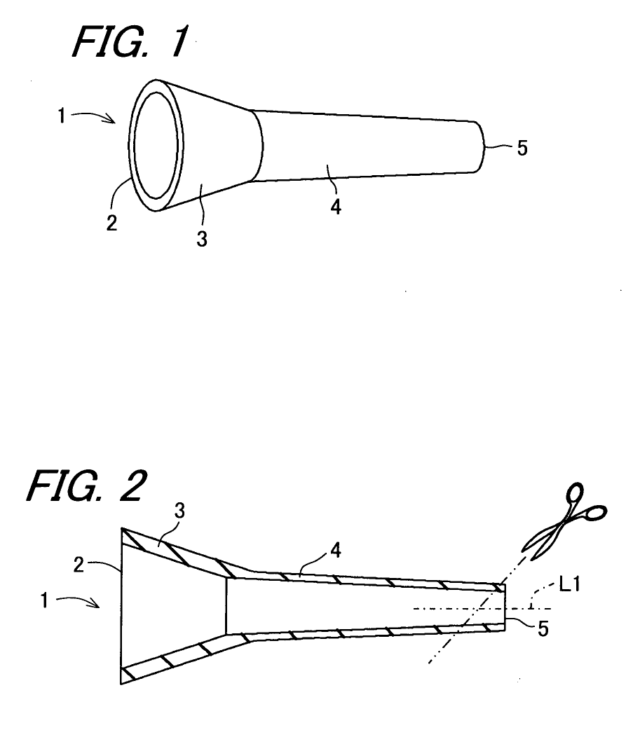

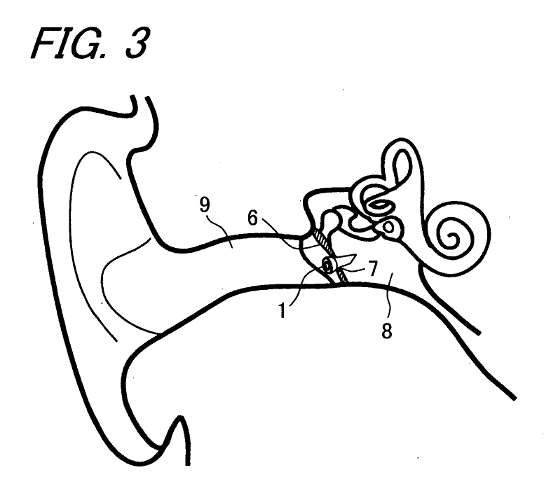

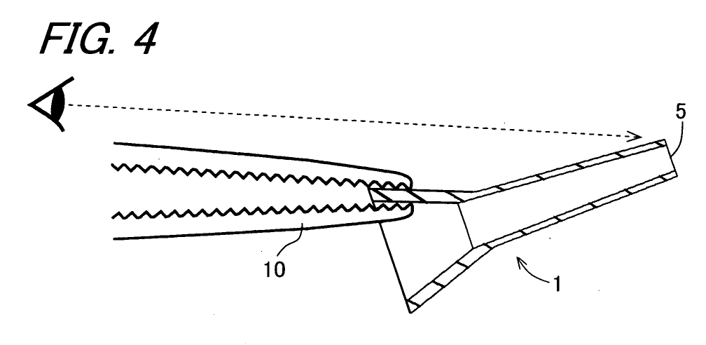

The tympanic membrane drain tube according to the invention will be described below with reference to the drawings. FIG. 1 is a perspective view illustrating a tympanic membrane drain tube according to a first embodiment of the invention. FIG. 2 is a cross sectional view illustrating the tympanic membrane drain tube according to the first embodiment of the invention. FIG. 3 is a view for explaining an inserted state of the tympanic membrane drain tube according to the first embodiment of the invention. And FIG. 4 is a view for explaining a pinched state of the tympanic membrane drain tube according to the first embodiment of the invention.

With reference to FIG. 1, a rear end 2 located on an external ear canal side of a hollow tube 4 made of a plastic material serving as a main body of a tympanic membrane drain tube 1 is provided with a flange portion 3 formed into an annular shape.

With reference to FIG. 1 and FIG. 2, the hollow tube 4 connected to the flange portion 3 is formed into a tapered shape of which inner and outer diameters on the rear end side are larger than inner and outer diameters of a tip end 5 and which is smaller in diameter toward the tip end 5.

Furthermore, an inner peripheral face of the hollow tube 4 is defined so that an angle formed by opposed lines of intersection, which lines are formed by two intersected planes; that is, an inner peripheral face defining a tapered internal space of the hollow tube 4 and a virtual plane including a diameter line of the hollow tube 4 and a central axis line of the hollow tube 4, is larger on the rear end 2 side than that on the tip end 5 side. That is to say, in the hollow tube 4, an expanding angle of the inner peripheral face on the rear end 2 side is larger than an expanding angle of the inner peripheral face on the tip end 5 side.

With reference to FIG. 2 and FIG. 3, the tympanic membrane drain tube 1 according to the invention is formed so as to be capable of being inserted into a stoma 7 of a tympanic membrane 6 by cutting the tip end 5 of the hollow tube 4 diagonally with respect to an axis line L1 of the hollow tube 4.

In FIG. 3, a reference numeral 8 denotes a middle ear cavity, and a reference numeral 9 denotes an external ear canal.

With reference to FIG. 3, the tympanic membrane drain tube 1 according to the invention is formed into a tapered shape and thus able to be attached to the tympanic membrane 6 only by forming a tiny hole in the tympanic membrane 6. The tympanic membrane drain tube 1 is small in size and thus able to be inserted to the hole with ease. The incision operation on the tympanic membrane 6 can be performed with ease and in a short time.

Further, with reference to FIG. 4, since an expanding angle of the inner peripheral face of the hollow tube 4 on the rear end 2 side is larger than an expanding angle of the inner peripheral face of the hollow tube 4 on the tip end 5 side, when pinching the rear end 2 of the tympanic membrane drain tube 1 with an instrument such as a dressing forceps 10, the tip end 5 of the tympanic membrane drain tube 1 turns slightly upward, in a consequence whereof operator's eyes, the flange portion 3 of the tympanic membrane drain tube 1, and the stoma 7 formed in incising the tympanic membrane are not arranged on the same visual axis so that the end of the tympanic membrane drain tube 1 is not brought under the forceps 10 and therefore not out of sight. Accordingly, the operator can visually check the position of the tip end 5 so that the tympanic membrane drain tube 1 can be inserted into the stoma 7 of the tympanic membrane 6 under clear vision. It is thus possible to keep high safety level.

With reference to FIG. 2, the tympanic membrane drain tube 1 may sometimes have to be inserted into the stoma 7 of the tympanic membrane 6 under bad conditions such that the external ear canal is curved; the external ear canal is narrow; and the tympanic membrane 6 is slanted at a position where the stoma 7 is formed. In such cases, the tympanic membrane drain tube 1 is treated with trimming the tip end 5 by use of scissors or the like to thereby sharpen the tip end 5. The sharpening of the tip end 5 of the tympanic membrane drain tube 1 facilitates position adjustment between the stoma 7 formed in the tympanic membrane 6 and the tip end 5 of the tympanic membrane drain tube 1, and moreover achieves small resistance in inserting the tympanic membrane drain tube 1, thus resulting in less stress upon the patient and the operator at the time of the insertion.

Furthermore, since the hollow tube 4 is made of a plastic material such as elastomer, silicone rubber, polytetrafluoroethylene, or polyethylene, the trimming can be performed easily with scissors or the like, and it is possible to adjust the hollow tube 4 in length and so on, on the spot in accordance with the age and symptom of the patient, the condition for inserting the tube, and the like factor.

Therefore, it is also possible to insert the drain tube 1 whose length is determined by taking into account a period before the drain tube 1 sheds due to foreign body exclusion action by a living body reaction of the patient and a period required for a remedy so that the drain tube 1 sheds at the same time when the patient recovers completely.

Besides, it is possible to remove the drain tube 1 only by pinching and pulling out the inserted drain tube 1 with an instrument, and there is almost no need of operation for the removal. Furthermore, by virtue of the tapered shape of the drain tube 1, even if the accumulated exudate flows into the drain tube 1 and is then dried to form a plug, the drain tube 1 can be removed without imposing a burden on the patient in order to clean the drain tube 1, and it is also easy to reinsert the drain tube 1.

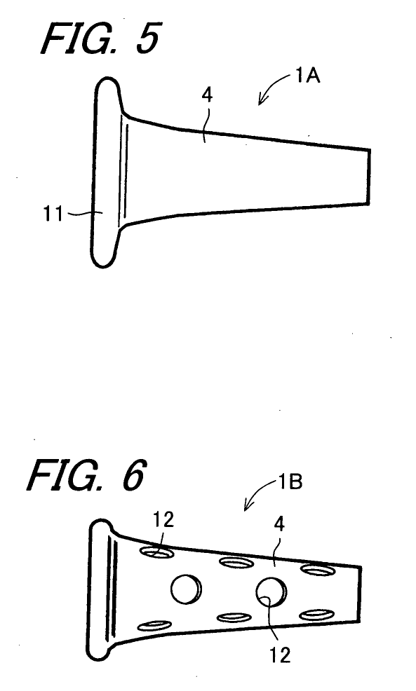

FIG. 5 is a front view illustrating a tympanic membrane drain tube 1A according to a second embodiment of the invention. The tympanic membrane drain tube 1A illustrated in FIG. 5 has the rear end 2 provided with an annular flange portion 11. In a case where the tympanic membrane drain tube 1A in this form is inserted into the tympanic membrane 6, it does not happen that the tympanic membrane drain tube 1A is excessively inserted by mistake, even if the stoma 7 is too large.

FIG. 6 is a front view illustrating a tympanic membrane drain tube 1B according to a third embodiment of the invention. The tympanic membrane drain tube 1B illustrated in FIG. 6 has the hollow tube 4 provided with a plurality of fine perforations 12. In a case where the tympanic membrane drain tube 1b in this form is attached to the tympanic membrane 6, a larger amount of air circulates from the external ear canal 9 to the middle ear cavity 8, whereby it is possible to further promote an effect of remedy. That is to say, the plurality of fine perforations 12 formed in the hollow tube 4 allows increase in amount of air passing through the tympanic membrane drain tube 1B without the need of increase in outer and inner diameters of the hollow tube 4.

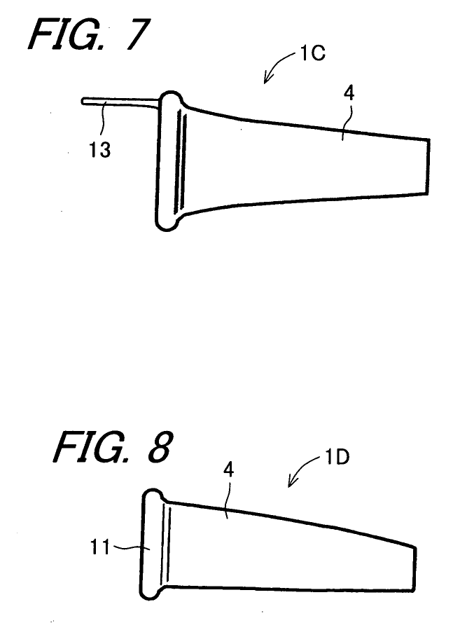

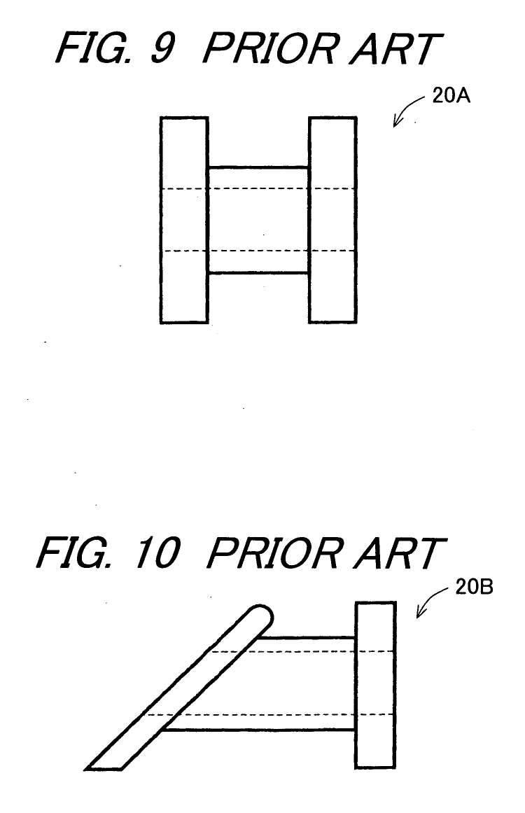

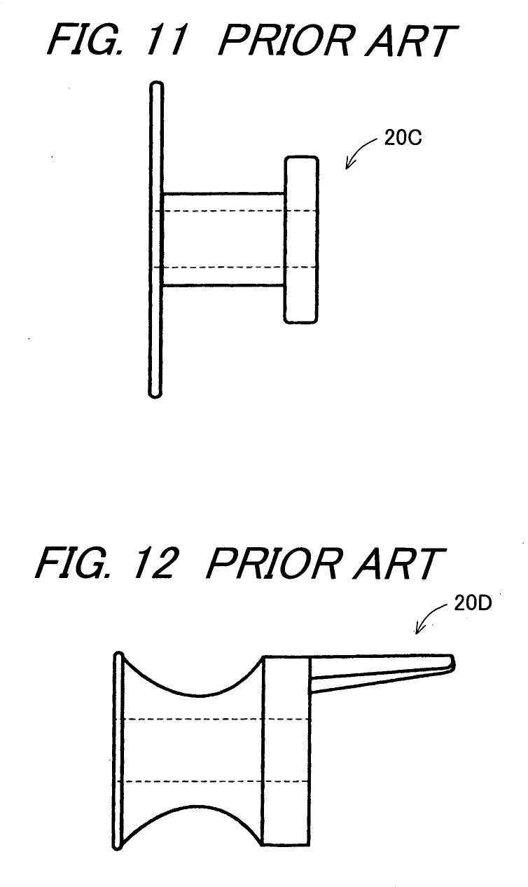

FIG. 7 is a front view illustrating a tympanic membrane drain tube 1C according to a fourth embodiment of the invention. A tongue-like assist tool 13 is attached to the rear end 2 of the hollow tube 4 in the tympanic membrane drain tube 1C illustrated in FIG. 7 so that the tympanic membrane drain tube 1c can be pinched easily with an instrument at the time of its removal. Consequently, it becomes possible to shorten a length of time to perform an operation for the removal of the tympanic membrane drain tube 1C.

FIG. 8 is a front view illustrating a tympanic membrane drain tube 1D according to a fifth embodiment of the invention. A lower side of the hollow tube 4 of the drain tube 1D is formed linearly, and an upper side on the opposite side thereof is formed into a gently curved tapered shape, whereby the hollow tube 4 is formed into a tapered shape.

Since the tympanic membrane 6 is not flat but curved by nature, an incised portion of the tympanic membrane 6 is not flat either. Therefore, the drain tube of the present embodiment can be inserted more easily than a drain tube formed into a simple tapered shape. Moreover, since exclusion action by a living body reaction during a remedy occurs smoothly, the drain tube can naturally shed in a short time.

As described above, attachment of the drain tube according to the invention greatly reduce a burden on the patient when compared with attachment of the conventional drain tube. Therefore, it is expected that the drain tube according to the invention is widely used for medical care in otorhinology, and the drain tube will be able to largely contribute to an improvement of medical technology.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

What is claimed is:

1. A tympanic membrane drain tube comprising a hollow tube,
wherein the hollow tube is formed so as to have a large diameter at a rear end and taper toward a front end, and
wherein the hollow tube can be safely inserted into a stoma formed in a tympanic membrane under clear vision.

2. The tympanic membrane drain tube of claim 1, wherein the hollow tube is made of a plastic material and can be trimmed.

3. The tympanic membrane drain tube of claim 1, wherein a flange portion is provided at the rear end of the hollow tube.

4. The tympanic membrane drain tube of claim 1, wherein the hollow tube has a fine perforation.

5. The tympanic membrane drain tube of claim 1, wherein a tongue-like assist tool is provided at the rear end of the hollow tube.

6. The tympanic membrane drain tube of claim 1, wherein the hollow tube has a section taken along its axial line which has one long side perpendicular to its short sides and another long side inclined toward the one long side and the another long side is slanted toward a tip end so as to form an inequable tapered shape.
